(19) United States
(12) Patent Application Publication
(10) Pub. No.: US 2003/0022241 A1
(43) Pub. Date: Jan. 30, 2003

(54) Methods

(76) Inventors: Christina Bendz, Ekero (SE); Staffan Lake, Lidingo (SE)

Correspondence Address: JANIS K. FRASER, PH.D., Fish & Richardson P.C., 225 Franklin Street, Boston, MA, 02110-2804, US

(21) Appl. No.: 10/174,784

(22) Filed: Jun. 18, 2002

(30) Foreign Application Priority Data
Jun. 18, 2001 (SE) 0102147-6

(51) Int. Cl. G01N 33/53; G01N 33/567

(52) U.S. Cl. 435/7.1; 435/7.21

(57) ABSTRACT

The invention relates to the use of the human “MAP kinase interacting kinases” Mnk2a or Mnk2b in methods for identification of pharmaceutically usefull agents, in particular agents useful for the treatment of type II diabetes. The invention also relates to methods of treating or preventing medical conditions relating to insulin resistance by modulating the expression or activity of Mnk2a or Mnk2b.

### CROSS REFERENCE TO RELATED APPLICATION

This application claims priority from Swedish Patent Application No. 0102147-6, filed Jun. 18, 2001, the entire content of which is incorporated herein by reference.

### TECHNICAL FIELD

The present invention relates to the use of the human “MAP kinase interacting kinases” Mnk2a and Mnk2b, in methods for identification of pharmaceutically useful agents, in particular agents useful for the treatment of type II diabetes.

### BACKGROUND ART

One of the major hormones that influences metabolism is insulin, which is synthesized in the beta cells of the islets of Langerhans of the pancreas. Insulin primarily regulates the direction of metabolism, shifting many processes toward the storage of substrates and away from their degradation (for reviews, see e.g. Shepherd, P. R. et al. (1998) Biochem. J. 333: 471-490; Alessi, D. R. & Downes, C. P. (1998) Biochim. Biophys. Acta 1436: 151-164). Insulin acts to increase the transport of glucose and amino acids as well as key minerals such as potassium, magnesium, and phosphate from the blood into cells. It also regulates a variety of enzymatic reactions within the cells, all of which have a common overall direction, namely the synthesis of large molecules from small units. A deficiency in the action of insulin (diabetes mellitus) causes severe impairment in (i) the storage of glucose in the form of glycogen and the oxidation of glucose for energy; (ii) the synthesis and storage of fat from fatty acids and their precursors and the completion of fatty-acid oxidation; and (iii) the synthesis of proteins from amino acids.

There are two varieties of diabetes. Type I is insulin-dependent diabetes mellitus (IDDM; formerly referred to as juvenile onset diabetes), for which insulin injection is required. In this type, insulin is not secreted by the pancreas and hence must be taken by injection. Type II diabetes, non-insulin-dependent diabetes mellitus (NIDDM), is characterized clinically by hyperglycemia and insulin resistance and is commonly associated with obesity. Type II diabetes is a heterogeneous group of disorders in which hyperglycemia results from both an impaired insulin secretory response to glucose and decreased insulin effectiveness in stimulating glucose uptake by skeletal muscle and in restraining hepatic glucose production (insulin resistance). Before diabetes develops, patients generally lose the early insulin secretory response to glucose and may secrete relatively large amounts of proinsulin. In established diabetes, although fasting plasma insulin levels may be normal or even increased in type II diabetes patients, glucose-stimulated insulin secretion is clearly decreased. The decreased insulin levels reduce insulin-mediated glucose uptake and fail to restrain hepatic glucose production.

Glucose homeostasis depends upon a balance between glucose production by the liver and glucose utilization by insulin-dependent tissues, such as fat and muscle, and insulin-independent tissues, such as brain and kidney. In type II diabetes, the entry of glucose into fat and muscle is reduced and glucose production in the liver is increased, due to insulin resistance in the tissues.

The receptor tyrosine kinases (RTKs) are a major type of cell-surface receptors. The ligands for RTKs are peptide/protein hormones including nerve growth factor (NGF), platelet-derived growth factor (PDGF), epidermal growth factor (EGF), and insulin. Binding of a ligand to an RTK stimulates the receptor's intrinsic protein-tyrosine kinase activity, which subsequently stimulates a signal-transduction cascade leading to changes in cellular physiology and patterns of gene expression. RTK signaling pathways have a wide spectrum of functions including regulation of cell proliferation and differentiation, promotion of cell survival, and modulation of cellular metabolism.

Ras is a GTP-binding switch protein that acts like a key signaling molecule in pathways triggered by activation of RTKs. All Ras-linked RTKs in mammalian cells appear to utilize a highly conserved signal-transduction pathway in which activated Ras induces a kinase cascade that culminates in the activation of MAP kinase (mitogen-activated protein kinase). This serine/threonine kinase, which can translocate into the nucleus, phosphorylates many different proteins including transcription factors that regulate expression of important cell-cycle and differentiation-specific proteins.

The murine Mnk1 and Mnk2 gene products (“MAP kinase interacting kinase” or “MAP kinase signal-integrating kinase” 1 and 2) are single-domain serine/threonine kinases that share 72% sequence identity (Waskiewicz A. J. et al. (1997) EMBO J. 16: 1909-1920; GenBank Accession Nos. Y11091 and Y11092). Human Mnk1 has also been described (Fukunaga, R. et al. (1999) EMBO J. 16: 1921-1933; GenBank Accession No. AB000409). All these three proteins were identified by their ability to bind tightly to MAP kinases. Both Mnk1 and 2 bind the extracellular signal-regulated kinases ERK1 and ERK2, and Mnk1 also binds the stress-activated kinase, p38. The eukaryotic initiation factor 4E (eIF4E) has been identified as one of the physiological substrates of Mnk1 and Mnk2 (Scheper, G. C. et al. (2001) Mol. Cell. Biol. 21: 743-754).

The human Mnk2 gene has been identified and characterized through a yeast two-hybrid screen in which the Mnk2 protein interacted with the ligand-binding domain of the estrogen receptor β (ERβ) (Slentz-Kesler, K. et al. (2000) Genomics 69: 63-71). It was shown that the human Mnk2 gene has two C-terminal splice variants, designated Mnk2a (the nucleotide and amino acid sequences of Mnk2a are designated SEQ ID NOS:1 and 2, respectively; GenBank Accession No. AF237775) and Mnk2b (the nucleotide and amino acid sequences of Mnk2b are designated SEQ ID NOS: 3 and 4, respectively; GenBank Accession No. AF237776). The two isoforms are identical over the first 385 amino acids of the coding sequence and differ only in the final exon which encodes an additional 80 residues for Mnk2a and 29 residues for Mnk2b. It was further shown that the Mnk2 interaction was selective for estrogen receptor (ER)β as opposed to ERα and that the interaction was specific to Mnk2b as opposed to Mnk2a or Mnkb 1.

### BRIEF DESCRIPTION OF THE DRAWINGS

FIG. 1 is a graph depicting the effect of Mnk2b overexpression in adipocytes 3T3-L1 transfected with GLU-REx3, CRE, IRE, or SREBP-RE. Control cells were transfected with an empty plasmid vector and the control expression level was set to 1.00.

FIG. 2 is a graph depicting the effect of Mnk2b on glucose uptake, when overexpressed in (2A) differentiated adipocytes 3T3-L1 and (2B) human neuronal cell line SHSY. Control cells (ctrl) were transfected with an empty plasmid vector. Grey staples indicate non-stimulated cells, white staples indicate insulin-stimulated cells.

FIG. 3 is a graph depicting the effect of Mnk2b overexpression and RNAi knock-down of Mnk2b expression on glucose uptake in human cells.

### DISCLOSURE OF THE INVENTION

It has surprisingly been found that Mnk2 is involved in the insulin-signaling pathway.

In one aspect, the invention features a method for identifying an agent that modulates (increases or decreases) the ability of an Mnk2 polypeptide to modulate glucose uptake in a cell, the method comprising: contacting an Mnk2 polypeptide with a candidate agent; and determining the effect of the candidate agent on the ability of the Mnk2 polypeptide to modulate glucose uptake in a cell. In one example, the candidate agent decreases the ability of the Mnk2 polypeptide to decrease glucose uptake in the cell.

In another aspect, the invention features a method for identifying an agent that modulates the ability of an Mnk2 polypeptide to modulate the activity of a glucose response element in a cell, the method comprising: contacting an Mnk2 polypeptide with a candidate agent; and determining the effect of the candidate agent on the ability of the Mnk2 polypeptide to modulate the activity of a glucose response element in a cell. In one example, the candidate agent decreases the ability of the Mnk2 polypeptide to decrease the activity of a glucose response element (e.g., a response element described herein) in the cell.

In another aspect, the invention features a method for identifying a modulator of glucose uptake, the method comprising: providing a cell expressing a recombinant Mnk2 polypeptide; exposing the cell to a candidate agent; and measuring glucose uptake in the cell in the presence of the candidate agent, wherein altered glucose uptake in the cell in the presence of the candidate agent compared to the absence of the candidate agent indicates that the candidate agent is a modulator of glucose uptake. In one example, the candidate agent causes increases glucose uptake.

A candidate agent can contain, for example, a peptide, peptidomimetic, amino acid, amino acid analog, polynucleotide, polynucleotide analog, nucleotide, nucleotide analog, or other small molecule. In one example, the candidate agent inhibits a Mnk2 biological activity such as a serine/threonine kinase activity, the ability to reduce glucose uptake in a cell, the ability to decrease activity of a glucose response element (e.g., an element described herein), and/or the ability to bind a Mnk2 ligand described herein. In one embodiment, the candidate agent binds to a Mnk2 polypeptide or a nucleic acid (RNA or DNA) encoding a Mnk2 polypeptide.

The screening methods described herein can optionally include a step of introducing into a cell a nucleic acid encoding a Mnk2 polypeptide. The effect of a candidate agent on a biological activity described herein can be evaluated in the presence and/or absence of a Mnk2 polypeptide or a nucleic acid encoding a Mnk2 polypeptide. The methods described herein can be carried out in vitro or in vivo using a cell-based system, a cell-free system, or a combination of cell-based and cell-free systems.

In another aspect, the invention features a method for modulating glucose uptake in a cell, the method comprising contacting a cell with an amount of a compound effective to modulate expression or activity of a Mnk2 polypeptide and thereby modulate glucose uptake in the cell.

In another aspect, the invention features a method for treating or preventing a medical condition relating to insulin resistance, the method comprising: selecting an individual that has or is at risk of having a medical condition relating to insulin resistance; and administering to the individual a compound that modulates expression or activity of an Mnk2 polypeptide in an amount effective to treat or prevent the medical condition.

A compound can be, for example, a candidate agent as described herein. In one embodiment, the compound decreases expression or activity of the Mnk2 polypeptide and thereby increases glucose uptake in the cell. For example, the compound can decrease kinase activity of the Mnk2 polypeptide.

The medical condition relating to insulin resistance can be associated with reduced glucose uptake. In one example, the medical condition is diabetes, e.g., type II diabetes.

The Mnk2 polypeptide used in the methods described herein can be a mammalian Mnk2 polypeptide, e.g., a human Mnk2 polypeptide. For example, the Mnk2 polypeptide can be a human Mnk2a or Mnk2b polypeptide.

The Mnk2 polypeptide can have a sequence shown as SEQ ID NO:2 or SEQ ID NO:4. A Mnk2 polypeptide can also differ from the corresponding sequence shown as SEQ ID NO:2 or SEQ ID NO:4. The differences are, preferably, differences or changes at a non-essential residue or a conservative substitution. In one embodiment, the Mnk2 polypeptide includes an amino acid sequence at least about 60% identical to a sequence shown as SEQ ID NO:2 or SEQ ID NO:4 or a fragment thereof. Preferably, the amino acid sequence is at least 65%, 70%, 75%, 80%, 85%, 90%, 95%, 98%, 99% or more identical to SEQ ID NO:2 or SEQ ID NO:4 and has a Mnk2 biological activity described herein. For example, the amino acid sequence can be identical to SEQ ID NO:2 or SEQ ID NO:4.

Preferred MnK2 polypeptides are at least 10%, preferably at least 20%, 30%, 40%, 50%, 60%, 70%, or more, of the length of the sequence shown as SEQ ID NO:2 or SEQ ID NO:4 and have a Mnk2 biological activity described herein. For example, a Mnk2 polypeptide can have a serine/threonine kinase activity, reduce glucose uptake in a cell, decrease activity of a glucose response element (e.g., an element described herein), and/or bind a Mnk2 ligand described herein.

A Mnk2 polypeptide also includes a polypeptide comprising a functional domain of the polypeptide of SEQ ID NO:2 or SEQ ID NO:4 described herein, e.g., a kinase domain. In one embodiment, the Mnk2 polypeptide has kinase activity.

A Mnk2 polypeptide also includes a polypeptide comprising at least 20, 30, 40, 50, 75, 100, 150, 200, 250, 300, 350, 400, 450, or more contiguous amino acid residues of SEQ ID NO:2 or SEQ ID NO:4. Preferably, the polypeptide has a Mnk2 biological activity described herein.

The Mnk2 polypeptide in some aspects of the invention can be a substantially pure polypeptide. The term “substantially pure” as used herein in reference to a given polypeptide means that the polypeptide is substantially free from other biological macromolecules. For example, the substantially pure polypeptide is at least 75%, 80, 85, 95, or 99% pure by dry weight. Purity can be measured by any appropriate standard method known in the art, for example, by column chromatography, polyacrylamide gel electrophoresis, or HPLC analysis.

Throughout this description the terms “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

Below, the invention is described in the appended examples, which are intended to illustrate the invention, without limiting the scope of protection.

### EXAMPLES

### Example 1

Identification of LK6 and Mnk

P element mediated mutagenesis is a widely used technology in Drosophila genetics (Cooley, L. et al. (1988) Science 239: 1121-1128; Robertson, H. M. et al. (1988) Genetics 118: 461-470). The P element is a well-characterized transposable element, which can introduce heritable loss of function mutations into a wide array of genes. Coupled with genomic annotation of the P element insertion site, P element libraries provide a valuable reverse genetics tool. Genetic screens using libraries of P insertion mutants in known genes enable a rapid scanning of the genome to identify potential modifier genes.

Impaired insulin receptor signaling have phenotypic manifestation of smaller cell size (Huang, H., et al. (1999) PTEN affects cell size, cell proliferation and apoptosis during Drosophila eye development. Development 126: 5365-5372.) A genetic screen was performed to identify modifiers of insulin receptor signaling, using a library of P insertion mutagenized Drosophila lines. In this screen, the small eye phenotypic manifestation was used as read out. The D. melanogaster gene LK6 (GenBank Accession No. U76378) was identified as a weak but consistent enhancer of the D.N. Insulin receptor phenotype.

The Drosophila melanogaster L6K protein was used in a TBLASTN search (http://www.ncbi.nlm.nih.gov/Educatioit/blasttutorial.htm1) in the public nucleotide databases (http://www.ncbi.nlm.nih.gov/blast/). The human hits were MNK1 (AB000409; e-value of e-113), MNK2a (AF237775; e-value of e-114) and MNK2b (AF237776; e-value of e-110). Consequently, it was concluded by bioinformatic analysis that the D. melanogaster gene LK6 has two human homologues, Mnk1 and Mnk2.

### Example 2

Cloning of MNK2b

The 3′-end of Mnk2b cDNA was isolated from Incyte clone No. 1309709. This clone contains a sequence corresponding to the last 570 bp of Mnk2b. The cDNA insert of the Incyte clone was verified by sequencing, performed by the ABI PrismBigDye Terminator Cycle Sequencing Ready Reaction Kit, on Applied Biosystems Model ABI 377 XL/96 DNA sequencing system.

To isolate a cDNA clone encoding the 5′-end of Mnk2b (670 bp), the public Mnk2b sequence (GenBank Accession No. AF237776) was used to design PCR primers for PCR amplification.

As template for cloning, cDNA made from human liver was used. A first strand cDNA synthesis, using SUPERSCRIPT Choice System (Life Technologies; Cat. # 18090-019) was performed using 1 μg human liver mRNA (Clontech; Cat. # 6510-1) with random hexamers according to the manufacturer's instructions.

The 100 μl PCR was performed using Native Pfu DNA Polymerase kit (Stratagene; Cat. # 600135) and 5 μl each of the gene specific primers LAKQ166 (SEQ ID NO: 5) and LAKQ168 (SEQ ID NO: 6). The Perkin Elmer DNA Thermal Cycler 480 was used with the program: 1 cycle of 94° C., 2 min; 60° C., 1 min; 74° C., 2 min; 25 cycles of 94° C., 1 min; 58° C., 1 min; 74° C., 2 min; and finally 72° C., 7 min followed by cooling to 4° C. Additional five rounds of amplification were performed as above, except that the annealing temperature was lowered to 55° C.

An aliquot, 15 μl, of the PCR was loaded on a 1% NuSieve GTG low melting temperature agarose gel (FMC BioProducts; Cat. #50082) and the fragment of about 670 bp was excised from the gel. 3 μl of the isolated fragment was cloned into 1 μl plasmid pCR2.1-TOPO using the TOPO TA Cloning Kit (Invitrogen; Cat. # K4500-01). 3 μl of the ligation mix was transformed into One Shot chemically competent TOP10 E. coli cells (Invitrogen; Cat. #C4040-03).

Plasmid DNA from three clones (3 ml overnight culture), were obtained by using the QIAprep Spin Miniprep Kit (QIAGEN; Cat. #27104) and the subsequent sequencing (#A0452) was performed as above. The plasmid with confirmed correct sequence was designated pMB 1500.

The two parts of Mnk2b, the 3′-end from Incyte1309709, and the 5′-end from pMB 1500, were joined together by a three-fragment ligation into pCR2.1-TOPO, yielding pBV1556.

2.4 μg of pBM1500 was digested with EcoRI and SacI, and 1.8 μg of the same plasmid was digested with EcoRI and BglII. Half of the digestions were loaded on a 1.2% E-Gel, Invitrogen, and a band of approximately 3800 bp (fragment a) was cut out from the EcoRI-SacI digestion, and a band of approximately 680 bp (fragment b) was cut out from the EcoRI-BglII digestion. 2.7 μg of the plasmid Incytel309709 was digested with SacI and BglII. Half of the digestion was loaded on an E-Gel and a band of approximately 700 bp (fragment c) was cut out from the gel. The fragments were purified using QIAquick Gel Extraction Kit (Qiagen; Cat. # 28704) and subsequent elution in 50 μl H₂O.

Ligation was performed using Ready-To-Go T4 DNA ligase (Amersham Pharmacia Biotech; Cat. # 27-0361-01). 6 μ; of fragment A was mixed with 7 μl of fragments B and C, respectively. The ligation mix was transformed into One Shot chemically competent Top10 E. coli cells, and plasmid DNA was obtained as above. Control digestions using the restriction enzymes used for cloning, EcoRI, SacI and BglII verified the insert.

Mnk2b for mammalian expression was made by using Gateway™ Cloning Technology from Life Technologies. Gateway compatible primers were designed (SEQ ID NOS: 7 and 8) and PCR was performed using pBV1556 as a template. The PCR was perfonned in 50 μl using Taq DNA Polymerase, Roche (Cat. # 1 435 094) and 1 μl each of the primers BEKA 248 and BEKA247. The Perkin Elmer Gene Amp PCR system 2400 was used with the following program: 95° C., 5 min; (95° C. 30 s, 55° C. 30 s, 72° C. 2 min)×25; and 72° C., 7 min; followed by cooling to 4° C. 10 μl of the reaction was loaded on a 1.2% E-Gel, and a fragment of approximately 1400 bp was cut out from the gel, and purified using QIAquick Gel Extraction Kit.

The PCR fragment was cloned into pDONR201 (Life Technologies; Cat. # 11798-014), according to the manufacturer's instructions. The resulting entry clone was designated pBV27, and the insert was confirmed by sequencing. A mammalian expression clone with 5′-GST fusion, designated pBV44 (SEQ ID NO: 9), was constructed (according to the manufacturer's instructions) using the destination vector pDEST27 (Life Technologies; Cat. # 11812-013). In SEQ ID NO: 9, amino acids 1 to 226 represents the GST domain, while amino acids 237 to 649 represents human Mnk2b.

### Example 3

Expression Profiling

To determine the relative expression levels of MNK2b in different tissues, a Multiple Tissue Expression Array (CLONTECH; Cat. #775) was used in a hybridization experiment with a 106 bp gene specific probe.

The MNK2b cDNA clone, pBV27, was digested with the restriction enzymes NcoI and PpuMI. The fragments were separated on a 1.2% agarose gel (Invitrogen; Cat. #G5018-01). ThelO6 bp fragment was exercised from the gel and purified using QIA quick Gel Extraction Kit (QIAGEN; Cat. #28704).

25 ng of the purified fragment was used in a ³²P labeling reaction, performed with reagents as recommended in the Strip-EZ DNA probe synthesis instruction manual (Ambion; Cat. #1470). [α-³²P]dATP used in the reaction was purchased from Amersham Pharmacia Biotech (Cat. #AA0004).

Hybridization and washing conditions were preformed as recommended by CLONTECH manual PT3307-1. The MTE Array was exposed in a STORM860 Phosphor Screen for 70 h. ImageQuant was used to analyze the hybridization signal.

The results indicated the highest expression levels for MNK2b in skeletal muscle. This was unexpected, since published results on adult mouse tissue showed expression of Mnk2 mRNA in all tissues studied, except for brain (Waskiewicz A. J. et al. (1997) EMBO J. 16: 1909-1920).

### Example 4

Overexpression of Mnk2b Affects Glucose Responsiveness and Lipid Metabolism in Mouse Adipocytes

Inducible reporter vectors that contain the Photinus pyralis (firefly) luciferase reporter gene, driven by a basic promoter element (TATA box), as well as inducible cis-enhancer elements (direct repeats from the promoter regions of various genes), were prepared or purchased. The reporter vectors are designed for the in vivo readouts of signal transduction pathways, since the enhancers are convergent points of many signal transduction pathways. When a plasmid expressing the gene of interest is cotransfected into mammalian cells with a cis-reporter plasmid, increased luciferase expression indicates either direct or indirect transcriptional activation.

A vector designated pGluREx3-Luciferase ((gtgCACGTGtgaCAGCTGcaa)x3; SEQ ID NO:10) was prepared using the pTAL promoter vector (Clontech; cat. #6252). The pGluREx3 vector is designed to monitor effects on glucose response (Portois L., et al. (1999) J. Biol. Chem. 274: 8181-8190).

A vector designated pSREBP-Luciferase (aTCACcCCAC; SEQ ID NO:11) was prepared by cloning two sterol regulatory element binding protein (SREBP) response elements into the pGLE2-promoter Vector (Promega; cat. #E1631). The pSREBP vector is designed to monitor effects on steroid response element (Yokyama, C. et al. (1993) Cell 75: 187-197).

The vector pCRE-Luciferase, designed to monitor the activation of cAMP binding protein (CREB) and cAMP-mediated signal transduction pathways, was purchased from Stratagene (cat. #219075).

A vector designated pIRE-Luciferase ((tagCAAAACAaactTATTTTGaaca)x3; SEQ ID NO:12) was prepared, using the pGL2-Promoter Vector (Promega; cat. #E1631). The pIRE vector is designed to monitor insulin receptor mediated signaling through the insulin-like growth factor binding protein (IGFBP-1).

Mouse adipocytes (differentiated 3T3-L1 cells) were transiently transfected with the response element construct of interest, in combination with Mnk2b or a backbone (control) plasmid construct, using LipofectAmine™2000 (Life Technologies). After 48 hrs, the cells were lysed using a lysis buffer (Tris-EDTA+0.25% Triton-X100) for 10 min at room temperature, and the luciferase activity was measured using a luciferase activity assay (BioThema).

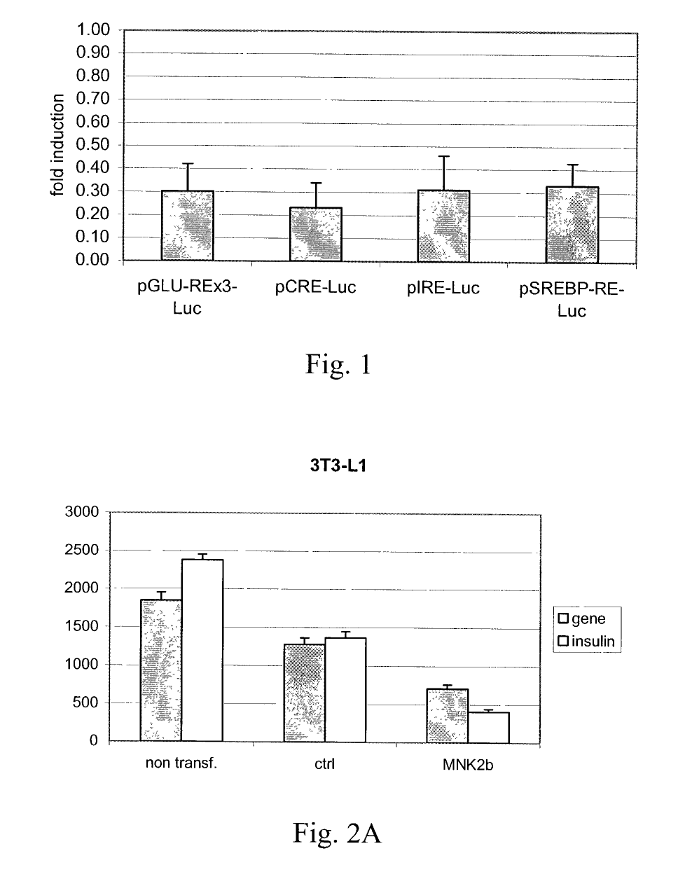

The results (FIG. 1) indicate that overexpression of Mnk2b in mouse adipocytes resulted in a 70% decrease of the activity of the GLUx3-Luciferase reporter, indicating a decrease in glucose responsiveness in the cells. To the inventors' knowledge, there are no previously disclosed results that indicate a link between Mnk2b and glucose uptake.

The results shown in FIG. 1 further indicate that overexpression of Mnk2b in mouse adipocytes leads to decreased activity of the SREBP response element. Our conclusion is that Mnk2b affects lipid metabolism, since the SREBP response element has been shown to control transcription of e.g. low density lipoprotein receptor gene (Yokoyama, C. et al. (1993) Cell 75: 187-197) and to regulate cholesterol metabolism (Brown, M. S. and Goldstein J. L. (1997) Cell 83: 331-340).

Overexpression of Mnk2b in mouse adipocytes also leads to decreased activity of the reporters pCRE and pIRE. These results confirm published data on Mnk2b as part of the MAP-kinase signaling pathway (Waskiewicz, A. et al. (1997) EMBO J. 16: 1909-1920; Fukunaga, R. and Hunter, T. (1997) EMBO J. 16: 1921-1933).

### Example 5

Overexpression of Mnk2b Modulates Glucose Uptake in Adipocytes

Glucose uptake was determined according to the method of Hundal et al. (1994) Biochem. J. 297: 289-295. Briefly, after incubation with hormones for 45 minutes, if not otherwise stated, cell monolayers were rinsed with glucose free PBS. Glucose uptake was quantified by incubating the cells in the presence of 1 μCi/ml ³H-2-deoxy-glucose in PBS for 8 min. Non-specific uptake was determined by quantifying cell-associated radioactivity in the presence of 10 μM cytochalasin B. Uptake of 2-deoxy-glucose was terminated by rapidly aspirating the medium, followed by three successive washes of cell monolayers with ice cold PBS. The cells were lysed in 0.5 M NaOH, followed by liquid scintillation counting. Rates of transport were normalized for protein content in each well.

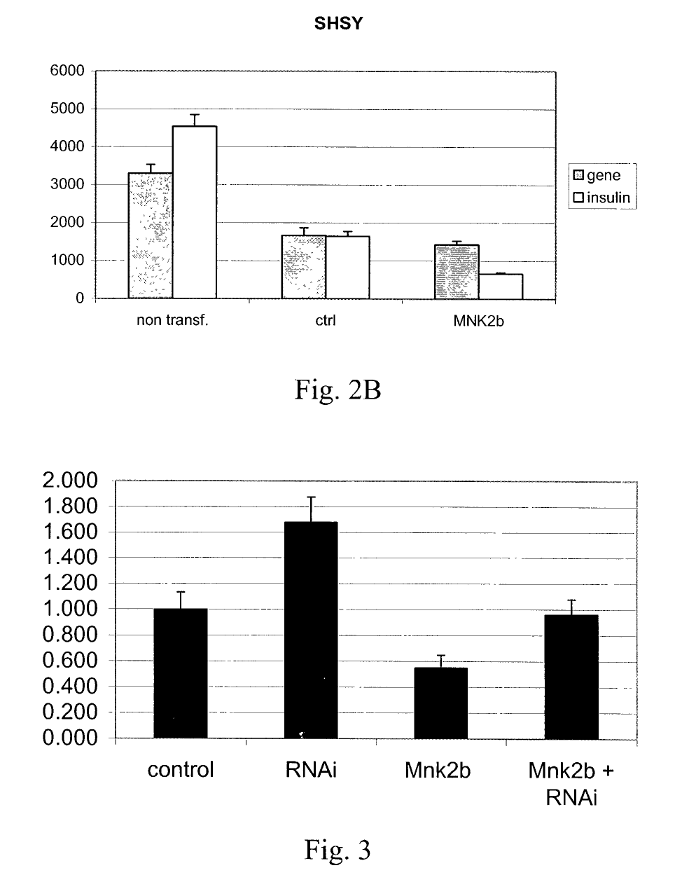

The results (FIG. 2) indicate that overexpression of Mnk2b in adipocytes (differentiated 3T3-L1 cells) and a human cell-line (SHSY) decreased the rate of glucose-uptake in an insulin-dependent manner. The results confirm the results from the reporter assay (Example 4) and further indicate that one effect of Mnk2b expression is reduction of glucose uptake.

### Example 6

Structure Models of Mnk Proteins

Three-dimensional structure models of Mnk1, Mnk2a and Mnt2b were prepared from homology data. The structure of rat calmodulin-dependent protein kinase (Protein Data Bank entry 1A06) was used as template for all three models. (The Protein Data Bank is available at http://www.rcsb.org/pdb; see also Berman et al. (2000) Nucleic Acids Research 28: 235-242). The structure models were prepared using the ICM software from MolSoft Inc. (http://www.molsoft.com).

The models of Mnk1, Mnk2a and Mnk2b were highly similar but some structural differences were identified, which might be employed to achieve binding selectivity. A number of 87 non-identical residues were identified when Mnk2b was compared to Mnk1. Many of those are situated away from the active site, but two interesting differences between Mnk1 and Mnk2b are Y→H in the active site (cf. position 95 in SEQ ID NO: 4) and T→L in a loop that could be involved in substrate recognition (cf. position 248 in SEQ ID NO: 4).

A comparison between Mnk2a and Mnk2b indicated that the C-terminus, which is the only part differing between the two splice variants, folds against the active site in the models. This indicates the possible to identify agents having specificity between Mnk2a and Mnk2b.

### Example 7

Knock-down of Mnk2 Modulates Glucose Response in Human Neuroblastoma Cells

RNAi (RNA interference) refers to the introduction of homologous double stranded RNA (dsRNA) to specifically target a gene's product, resulting in null or hypomorphic phenotypes. RNAi technique was used to study effects on glucose response in cultivated cells upon knock-down of Mnk2 protein expression. Human neuroblastoma (SH-SY5y) cells were transiently transfected with a glucose response element coupled to a luciferase reporter gene (GluREx3-Luciferase), Mnk2b or backbone plasmid and [RNAi-Mnk2] using LipofectAmine2000 (LifeTechnologies). For each well in a 96 well plate 0.2 μg GluREx3-Luciferase, 0.07 μg Mnk2b/backbone and 0.13 μg [RNAi-Mnk2] were mixed with 1.8 μl LA2000/ug DNA diluted in 50 μl Opti-MEM (Gibco). After 48 h. the cells were lysed using 15 μl/well lysis buffer (TRIS-EDTA with 0.25% Triton x100) and the luciferase activity was measured (Luciferase activity assay kit, BioThema).

The results (FIG. 3) indicate that knock-down of Mnk2 protein expression in human neuroblastoma (SH-SY5y) cells by the use of RNAi leads to an increase in the activity of the glucose-response element. Over-expression of Mnk2b protein in the same cells, decreases the activity of the glucose-response element. This decrease is neutralized by knock-down of the over-expressed Mnk2b protein, that is combined transfection of expression plasmid and RNAi in the same cells.

### Example 8

NMR Screening for Compounds Binding Mnk2b

A diversity library consisting of relatively small and highly water-soluble compounds was used to screen native MNK-2B for binders by NMR (Nuclear Magnetic Resonance). The NMR tecinique used to identify binders was Saturation Transfer Difference (STD): the protein ¹H resonances are saturated by means of a weak radio-frequency field applied to a narrow spectral region. The saturation is transferred by spin diffusion to the rest of the protein and subsequently further to compounds that bind to the protein attenuating their signals in the NMR spectrum. The spectrum is then subtracted from a spectrum obtained at non-saturating conditions to obtain an STD spectrum showing only the signals from compounds interacting with the protein. In practice the pulse sequence is written in such a way that the subtraction is done automatically in every other scan, i.e. the individual spectra are never observed (Mayer & Meyer, Angew. Chem. Int. Ed., 38, 1784-1788, 1999).

The compounds in the library are divided into mixtures consisting of 4-8 compounds each. Each sample contained 1 μM native MNK-2B, 200 μM compound mixture, 50 mM sodium phosphate buffer, 1 mM DTT, pH 7.5 in ca 98% D₂O/2% H₂O. One sample did not contain any compounds and functioned as a negative control. The volume was 600 μl and standard NMR tubes were used. Experiments were performed on a 600 MHz Varian Unity NMR spectrometer at 20° C. A reference ¹H 1D experiment and an STD experiment were recorded on each sample. The binders identified from the screen were rerun as a single compound for confirmation. For these follow-up experiments samples containing 2 μM native Mnk2B and 250 μM of the individual compound were used. Several compounds, for instance 4-hydroxy-benzoic acid methyl ester, were identified as Mnk2b ligands.

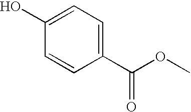

### 4-Hydroxy-benzoic acid methyl ester

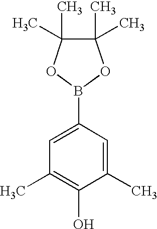

A kinase activity assay according to standard methods indicated that the identified compounds inhibited Mnk2b kinase activity in a dose-dependent manner. Consequently, it was shown that it is possible to identify small compounds acting as Mnk2b ligands.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A method for identifying an agent that modulates the ability of an Mnk2 polypeptide to modulate glucose uptake in a cell, the method comprising:
contacting an Mnk2 polypeptide with a candidate agent; and
determining the effect of the candidate agent on the ability of the Mnk2 polypeptide to modulate glucose uptake in a cell.

2. The method of claim 1, wherein the Mnk2 polypeptide is a mammalian Mnk2 polypeptide.

3. The method of claim 2, wherein the mammalian Mnk2 polypeptide is a human Mnk2 polypeptide.

4. The method of claim 2, wherein the mammalian Mnk2 polypeptide is Mnk2a.

5. The method of claim 4, wherein the Mnk2a polypeptide comprises the amino acid sequence of SEQ ID NO:2.

6. The method of claim 2, wherein the mammalian Mnk2 polypeptide is Mnk2b.

7. The method of claim 6, wherein the Mnk2b polypeptide comprises the amino acid sequence of SEQ ID NO:4.

8. A method for identifying an agent that modulates the ability of an Mnk2 polypeptide to modulate the activity of a glucose response element in a cell, the method comprising:
contacting an Mnk2 polypeptide with a candidate agent; and
determining the effect of the candidate agent on the ability of the Mnk2 polypeptide to modulate the activity of a glucose response element in a cell.

9. The method of claim 8, wherein the Mnk2 polypeptide is a mammalian Mnk2 polypeptide.

10. The method of claim 9, wherein the mammalian Mnk2 polypeptide is a human Mnk2 polypeptide.

11. The method of claim 9, wherein the mammalian Mnk2 polypeptide is Mnk2a.

12. The method of claim 11, wherein the Mnk2a polypeptide comprises the amino acid sequence of SEQ ID NO:2.

13. The method of claim 9, wherein the mammalian Mnk2 polypeptide is Mnk2b.

14. The method of claim 13, wherein the Mnk2b polypeptide comprises the amino acid sequence of SEQ ID NO:4

15. A method for identifying a modulator of glucose uptake, the method comprising:
providing a cell expressing a recombinant Mnk2 polypeptide;
exposing the cell to a candidate agent; and
measuring glucose uptake in the cell in the presence of the candidate agent,
wherein altered glucose uptake in the cell in the presence of the candidate agent compared to the absence of the candidate agent indicates that the candidate agent is a modulator of glucose uptake.

16. A method for modulating glucose uptake in a cell, the method comprising contacting a cell with an amount of a compound effective to modulate expression or activity of a MnK2 polypeptide and thereby modulate glucose uptake in the cell.

17. The method of claim 16, wherein the compound decreases expression or activity of the Mnk2 polypeptide and thereby increases glucose uptake in the cell.

18. The method of claim 17, wherein the compound decreases kinase activity of the Mnk2 polypeptide.

19. A method for treating or preventing a medical condition relating to insulin resistance, the method comprising:
selecting an individual that has or is at risk of having a medical condition relating to insulin resistance; and
administering to the individual a compound that modulates expression or activity of an Mnk2 polypeptide in an amount effective to treat or prevent the medical condition.

20. The method of claim 19, wherein the medical condition relating to insulin resistance is associated with reduced glucose uptake.

21. The method of claim 19, wherein the medical condition relating to insulin resistance is type II diabetes.


SEQUENCE LISTING (12 sequences)

SEQ ID NO: 1 - DNA, 1444 bp, Homo sapiens
  CDS (37)...(1431)
cggtcccctcccccgctggcggggcccggacagaagatggtgcagaagaaaccagccgaacttcagggtttccaccgttcgttcaaggggcagaaccccttcgagctggccttctccctagaccagcccgaccacggagactctgactttggcctgcagtgctcagcccgccctgacatgcccgccagccagcccattgacatcccggacgccaagaagaggggcaagaagaagaagcgcggccgggccaccgacagcttctcgggcaggtttgaagacgtctaccagctgcaggaagatgtgctgggggagggcgctcatgcccgagtgcagacctgcatcaacctgatcaccagccaggagtacgccgtcaagatcattgagaagcagccaggccacattcggagcagggttttcagggaggtggagatgctgtaccagtgccagggacacaggaacgtcctagagctgattgagttcttcgaggaggaggaccgcttctacctggtgtttgagaagatgcggggaggctccatcctgagccacatccacaagcgccggcacttcaacgagctggaggccagcgtggtggtgcaggacgtggccagcgccttggactttctgcataacaaaggcatcgcccacagggacctaaagccggaaaacatcctctgtgagcaccccaaccaggtctcccccgtgaagatctgtgacttcgacctgggcagcggcatcaaactcaacggggactgctcccctatctccaccccggagctgctcactccgtgcggctcggcggagtacatggccccggaggtagtggaggccttcagcgaggaggctagcatctacgacaagcgctgcgacctgtggagcctgggcgtcatcttgtatatcctactcagcggctacccgcccttcgtgggccgctgtggcagcgactgcggctgggaccgcggcgaggcctgccctgcctgccagaacatgctgtttgagagcatccaggagggcaagtacgagttccccgacaaggactgggcccacatctcctgcgctgccaaagacctcatctccaagctgctggtccgtgacgccaagcagaggctgagtgccgcccaagtcctgcagcacccctgggttcaggggtgcgccccggagaacaccttgcccactcccatggtcctgcagaggaacagctgtgccaaagacctcacgtccttcgcggctgaggccattgccatgaaccggcagctggcccagcacgacgaggacctggctgaggaggaggccgcggggcagggccagcccgtcctggtccgagctacctcacgctgcctgcagctgtctccaccctcccagtccaagctggcgcagcggcggcaaagggccagtctgtcctcggccccagtggtcctggtgggagaccacgcctgaccctcccatc

SEQ ID NO: 2 - protein, 465 aa, Homo sapiens
MVQKKPAELQGFHRSFKGQNPFELAFSLDQPDHGDSDFGLQCSARPDMPASQPIDIPDAKKRGKKKKRGRATDSFSGRFEDVYQLQEDVLGEGAHARVQTCINLITSQEYAVKIIEKQPGHIRSRVFREVEMLYQCQGHRNVLELIEFFEEEDRFYLVFEKMRGGSILSHIHKRRHFNELEASVVVQDVASALDFLHNKGIAHRDLKPENILCEHPNQVSPVKICDFDLGSGIKLNGDCSPISTPELLTPCGSAEYMAPEVVEAFSEEASIYDKRCDLWSLGVILYILLSGYPPFVGRCGSDCGWDRGEACPACQNMLFESIQEGKYEFPDKDWAHISCAAKDLISKLLVRDAKQRLSAAQVLQHPWVQGCAPENTLPTPMVLQRNSCAKDLTSFAAEAIAMNRQLAQHDEDLAEEEAAGQGQPVLVRATSRCLQLSPPSQSKLAQRRQRASLSSAPVVLVGDHA

SEQ ID NO: 3 - DNA, 1564 bp, Homo sapiens
  CDS (37)...(1278)
cggtcccctcccccgctggcggggcccggacagaagatggtgcagaagaaaccagccgaacttcagggtttccaccgttcgttcaaggggcagaaccccttcgagctggccttctccctagaccagcccgaccacggagactctgactttggcctgcagtgctcagcccgccctgacatgcccgccagccagcccattgacatcccggacgccaagaagaggggcaagaagaagaagcgcggccgggccaccgacagcttctcgggcaggtttgaagacgtctaccagctgcaggaagatgtgctgggggagggcgctcatgcccgagtgcagacctgcatcaacctgatcaccagccaggagtacgccgtcaagatcattgagaagcagccaggccacattcggagcagggttttcagggaggtggagatgctgtaccagtgccagggacacaggaacgtcctagagctgattgagttcttcgaggaggaggaccgcttctacctggtgtttgagaagatgcggggaggctccatcctgagccacatccacaagcgccggcacttcaacgagctggaggccagcgtggtggtgcaggacgtggccagcgccttggactttctgcataacaaaggcatcgcccacagggacctaaagccggaaaacatcctctgtgagcaccccaaccaggtctcccccgtgaagatctgtgacttcgacctgggcagcggcatcaaactcaacggggactgctcccctatctccaccccggagctgctcactccgtgcggctcggcggagtacatggccccggaggtagtggaggccttcagcgaggaggctagcatctacgacaagcgctgcgacctgtggagcctgggcgtcatcttgtatatcctactcagcggctacccgcccttcgtgggccgctgtggcagcgactgcggctgggaccgcggcgaggcctgccctgcctgccagaacatgctgtttgagagcatccaggagggcaagtacgagttccccgacaaggactgggcccacatctcctgcgctgccaaagacctcatctccaagctgctggtccgtgacgccaagcagaggctgagtgccgcccaagtcctgcagcacccctgggttcaggggtgcgccccggagaacaccttgcccactcccatggtcctgcagaggtgggacagtcacttcctcctccctccccacccctgtcgcatccacgtgcgacctggaggactggtcagaaccgttactgtgaatgagtgaagatcctggaggaccctgggccccaggccagctcccatcgctgggggacggtgaacggccatgtgttaatgttacgatgtttttaaaagacaaaaaaaaaaaaaaaacctcaaaagtttttttaaagtgggggaaaaacatccaagcactttaattccaatgtaccaggtgaactgacggagctcagaagttttcctttacaccaactgtcaatgccggaattttgtattctgttttgtaaagatttaataaaagtcaaaaaacttgcaaaaaaaaaaaaaaa

SEQ ID NO: 4 - protein, 414 aa, Homo sapiens
MVQKKPAELQGFHRSFKGQNPFELAFSLDQPDHGDSDFGLQCSARPDMPASQPIDIPDAKKRGKKKKRGRATDSFSGRFEDVYQLQEDVLGEGAHARVQTCINLITSQEYAVKIIEKQPGHIRSRVFREVEMLYQCQGHRNVLELIEFFEEEDRFYLVFEKMRGGSILSHIHKRRHFNELEASVVVQDVASALDFLHNKGIAHRDLKPENILCEHPNQVSPVKICDFDLGSGIKLNGDCSPISTPELLTPCGSAEYMAPEVVEAFSEEASIYDKRCDLWSLGVILYILLSGYPPFVGRCGSDCGWDRGEACPACQNMLFESIQEGKYEFPDKDWAHISCAAKDLISKLLVRDAKQRLSAAQVLQHPWVQGCAPENTLPTPMVLQRWDSHFLLPPHPCRIHVRPGGLVRTVTVNE

SEQ ID NO: 5 - DNA, 28 bp, Homo sapiens
atggtgcagaagaaaccagccgaacttc

SEQ ID NO: 6 - DNA, 27 bp, Homo sapiens
gcccaggtcgaagtcacagatcttcac

SEQ ID NO: 7 - DNA, 49 bp, Homo sapiens
ggggacaagtttgtacaaaaaagcaggcttcgtgcagaagaaaccagcc

SEQ ID NO: 8 - DNA, 54 bp, Homo sapiens
ggggaccactttgtacaagaaagctgggtcctactcattcacagtaacggttct

SEQ ID NO: 9 - protein, 649 aa, Homo sapiens
  DOMAIN (1)...(226)
  DOMAIN (237)...(649)
MAPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRSRSTSLYKKAGFVQKKPAELQGFHRSFKGQNPFELAFSLDQPDHGDSDFGLQCSARPDMPASQPIDIPDAKKRGKKKKRGRATDSFSGRFEDVYQLQEDVLGEGAHARVQTCINLITSQEYAVKIIEKQPGHIRSRVFREVEMLYQCQGHRNVLELIEFFEEEDRFYLVFEKMRGGSILSHIHKRRHFNELEASVVVQDVASALDFLHNKGIAHRDLKPENILCEHPNQVSPVKICDFDLGSGIKLNGDCSPISTPELLTPCGSAEYMAPEVVEAFSEEASIYDKRCDLWSLGVILYILLSGYPPFVGRCGSDCGWDRGEACPACQNMLFESIQEGKYEFPDKDWAHISCAAKDLISKLLVRDAKQRLSAAQVLQHPWVQGCAPENTLPTPMVLQRWDSHFLLPPHPCRIHVRPGGLVRTVTVNE

SEQ ID NO: 10 - DNA, 63 bp, Artificial Sequence
  vector
gtgcacgtgtgacagctgcaagtgcacgtgtgacagctgcaagtgcacgtgtgacagctgcaa

SEQ ID NO: 11 - DNA, 10 bp, Artificial Sequence
  vector
atcaccccac

SEQ ID NO: 12 - DNA, 75 bp, Artificial Sequence
  vector
tagcaaaacaaacttattttgaacatagcaaaacaaacttattttgaacatagcaaaacaaacttattttgaaca